(19) United States
(12) Patent Application Publication
(10) Pub. No.: US 2008/0177127 A1
(43) Pub. Date: Jul. 24, 2008

(54) Asymmetric Radiation Dosing Devices and Methods for Brachytherapy

(76) Inventors: Donna Allan, Bedford, NH (US); Greg Amante, Holliston, MA (US); Risa Anav, San Jose, CA (US); Maria Benson, West Boylston, MA (US); Michael Crocker, Half Moon Bay, CA (US); Gregory K. Edmundson, Rough & Ready, CA (US); Friedrick Ho, Mountain View, CA (US); Robert Kotmel, Burlingame, CA (US); Glenn Magnuson, Littleton, MA (US); Gregory T. Martin, Somerville, MA (US); Walter Ocampo, Marlborough, MA (US); Mukund Patel, San Jose, CA (US); F. Mark Payne, Palo Alto, CA (US); Russel Sampson, Palo Alto, CA (US); Jessica Schenck, Shrewsbury, MA (US); James Stubbs, Palo Alto, CA (US); Moshe Zilversmit, Mountain View, CA (US)

Correspondence Address: Heather Larson;Cytyc Corporation, 250 Campus Drive, Marlborough, MA, 01752, US

(21) Appl. No.: 11/959,252

(22) Filed: Dec. 18, 2007

Related U.S. Application Data
(60) Provisional application No. 60/870,670, filed on Dec. 19, 2006.

(51) Int. Cl. A61M 36/04

(52) U.S. Cl. 600/7

(57) ABSTRACT

A brachytherapy treatment device includes at least one tubular insertion member, an expandable member, and means for deflecting the at least one tubular insertion member. The tubular insertion member has a longitudinal axis and proximal and distal ends. The expandable member is disposed on and surrounding the distal end of the tubular insertion member. The distal end of the at least one deflected tubular insertion member within the first expandable member is offset from the longitudinal axis when deflected. The at least one deflected tubular insertion member is configured to receive a radiation source to position a radiation source offset with regard to the longitudinal axis to form an asymmetric radiation dosing profile. Additional brachytherapy treatment devices and methods for forming an asymmetric radiation dosing profile are disclosed.

### CROSS-REFERENCE TO RELATED APPLICATION

This application claims priority to U.S. Provisional Application Ser. No. 60/870,670, filed on Dec. 19, 2006 and entitled “Asymmetric Radiation Dosing Devices and Methods,” which is incorporated by reference herein for all that it discloses.

### TECHNICAL FIELD

This technology relates generally to brachytherapy devices and methods for use in treating proliferative tissue disorders.

### BACKGROUND

Body tissues subject to proliferative tissue disorders, such as malignant tumors, are often treated by surgical resection of the tumor to remove as much of the tumor as possible. Unfortunately, the infiltration of the tumor cells into normal tissues surrounding the tumor may limit the therapeutic value of surgical resection because the infiltration can be difficult or impossible to treat surgically. Radiation therapy may be used to supplement surgical resection by targeting the residual tumor margin after resection, with the goal of reducing its size or stabilizing it. Radiation therapy may be administered through one of several methods, or a combination of methods, such as interstitial or intercavity brachytherapy. Brachytherapy uses a source of radiation from seeds that contain radioactive isotopes and/or may also be administered via electronic sources that emit x-rays, for example.

Brachytherapy is radiation therapy in which the source of radiation is placed in or close to the area to be treated, such as within a cavity or void left after surgical resection of a tumor. Brachytherapy may be administered by implanting or delivering a spatially confined radioactive material to a treatment site, which may be a cavity left after surgical resection of a tumor. For example, brachytherapy may be performed by using an implantable device (e.g., catheter or applicator) to implant or deliver radiation sources directly into the tissue(s) or cavity to be treated. During brachytherapy treatment, a catheter may be inserted into the body at or near the treatment site and subsequently a radiation source may be inserted through the catheter and placed at the treatment site.

Brachytherapy is typically most appropriate where: 1) malignant tumor regrowth occurs locally, within 2 or 3 cm of the original boundary of the primary tumor site; 2) radiation therapy is a proven treatment for controlling the growth of the malignant tumor; and 3) there is a radiation dose-response relationship for the malignant tumor, but the dose that can be given safely with conventional external beam radiotherapy is limited by the tolerance of normal tissue. Interstitial and/or intercavity brachytherapy may be useful for treating malignant brain and breast tumors, among other types of proliferative tissue disorders.

There are two basic types of brachytherapy, high dose rate and low dose rate. These types of brachytherapy generally include the implantation of radioactive “seeds,” such as palladium or iodine, into the tumor, organ tissues, or cavity to be treated. Low dose rate (LDR) brachytherapy refers to placement of multiple sources (similar to seeds) in applicators or catheters, which are themselves implanted in a patient's body. These sources are left in place continuously over a treatment period of several days, after which both the sources and applicators are removed. High dose rate brachytherapy (HDR) uses catheters or applicators similar to those used for LDR. Typically, only a single radiation source is used, but of very high strength. This single source is remotely positioned within the applicators at one or more positions, for treatment times which are measured in seconds to minutes. The treatment is divided into multiple sessions (‘fractions’), which are repeated over a course of a few days. In particular, an applicator (also referred to as an applicator catheter or treatment catheter) is inserted at the treatment site so that the distal region is located at the treatment site while the proximal end of the applicator protrudes outside the body. The proximal end is connected to a transfer tube, which in turn is connected to an afterloader to create a closed transfer pathway for the radiation source to traverse. Once the closed pathway is complete, the afterloader directs its radioactive source (which is attached to the end of a wire controlled by the afterloader) through the transfer tube into the treatment applicator for a set amount of time. When the treatment is completed, the radiation source is retracted back into the afterloader, and the transfer tube is disconnected from the applicator.

A typical applicator catheter comprises a tubular member having a distal portion which is adapted to be inserted into the patient's body, and a proximal portion which extends outside of the patient. A balloon is provided on the distal portion of the tubular member which, when placed at the treatment site and inflated, causes the surrounding tissue to substantially conform to the surface of the balloon. In use, the applicator catheter is inserted into the patient's body, for instance, at the location of a surgical resection to remove a tumor. The distal portion of the tubular member and the balloon are placed at, or near, the treatment site, e.g. the resected space. The balloon is inflated, and a radiation source is placed through the tubular member to the location within the balloon.

Several brachytherapy devices are described in U.S. Provisional Patent Application 60/870,690, entitled “Brachytherapy Device and Method,” filed on Dec. 19, 2006, and U.S. Provisional Patent Application 60/870,670, entitled “Asymmetric Radiation Dosing Devices and Methods,” filed on Dec. 19, 2006, and in copending U.S. Patent Application entitled “Selectable Multi-Lumen Brachytherapy Devices and Methods,” filed on or about Dec. 18, 2007, which are all commonly owned with the present application, U.S. Pat. No. 5,913,813, and U.S. Pat. No. 6,482,142, all of which are hereby incorporated by reference herein in their entireties.

The dose rate at a target point exterior to a radiation source is inversely proportional to the square of the distance between the radiation source and the target point. Thus, previously described applicators, such as those described in U.S. Pat. No. 6,482,142, issued on Nov. 19, 2002, to Winkler et al., are symmetrically disposed about the axis of the tubular member so that they position the tissue surrounding the balloon at a uniform or symmetric distance from the axis of the tubular member. In this way, the radiation dose profile from a radiation source placed within the tubular member at the location of the balloon is symmetrically shaped relative to the balloon. In general, the amount of radiation desired by a treating physician is a certain minimum amount that is delivered to a region up to about two centimeters away from the wall of the excised tumor, i.e. the target treatment region. It is desirable to keep the radiation that is delivered to the tissue in this target tissue within a narrow absorbed dose range to prevent over-exposure to tissue at or near the balloon wall, while still delivering the minimum prescribed dose at the maximum prescribed distance from the balloon wall (i.e. the two centimeter thickness surrounding the wall of the excised tumor).

However, in some situations, such as a treatment site located near sensitive tissue like a patient's skin, the symmetric dosing profile may provide too much radiation to the sensitive tissue such that the tissue suffers damage or even necrosis. In such situations, the dosing profile may cause unnecessary radiation exposure to healthy tissue or it may damage sensitive tissue, or it may not even be possible to perform a conventional brachytherapy procedure.

T o alleviate some of these problems associated with prior applicators, an asymmetric dosing profile is produced by shaping or locating the radiation source so as to be asymmetrically placed with respect to the longitudinal axis of the balloon. In an alternative approach, the applicator is provided with asymmetric radiation shielding located between the radiation source and the target tissue.

However, asymmetrically placing the radiation source decreases the radiation dosing profile in certain directions, but correspondingly increases the radiation dosing profile in the other directions. Some devices may not allow for adjustment of the amount of asymmetry and/or the resulting radiation dosing profile shape. Accordingly, there remains a need for additional methods and devices which can provide an asymmetric radiation dosing profile having a predetermined orientation during brachytherapy procedures.

### SUMMARY

Brachytherapy treatment devices and methods are disclosed herein. The brachytherapy treatment devices and methods disclosed herein may be oriented to create an asymmetric radiation dosing profile relative to an inner boundary of target tissue at a treatment site. The asymmetric radiation dosing profile functions to protect certain sensitive tissues from receiving an undesirably high dose of radiation while still allowing the remainder of target tissue at a treatment site to receive a prescribed therapeutic dosage of radiation treatment.

In one embodiment, a brachytherapy treatment device has at least one tubular insertion member, a first expandable member, and a means for deflecting the at least one tubular insertion member. The at least one tubular insertion member has a longitudinal axis, a proximal end and a distal end. The first expandable member is disposed on and surrounding the distal end of the tubular insertion member. The distal end of the at least one tubular insertion member within the first expandable member is offset from the longitudinal axis when deflected. The at least one deflected tubular insertion member is configured to receive a radiation source to position a radiation source offset with regard to the longitudinal axis to form an asymmetric radiation dosing profile.

The means for deflecting the at least one tubular insertion member may include, but is not limited to: differential wall thicknesses; differential materials having differing durometer, column strength, or shape memory properties; pull-wires; threaded members such as turnbuckles or lead screws; a pre-stressed or pre-bent member; a second expandable member; a slide mechanism; a helical-shaped member; detachable proximal and distal tip segments; an insertion support structure adjacent to the tubular insertion member; and/or an adjustable radiation source position mechanism.

In another embodiment, a brachytherapy treatment device includes at least one tubular insertion member and an expandable member. The at least one tubular insertion member has a longitudinal axis, a proximal end and a distal end. The expandable member is disposed on and surrounds the distal end of the at least one tubular insertion member. The distal end of the at least one tubular insertion member within the expandable member has a substantially helical shape. The at least one helical-shaped tubular insertion member is operable to receive a radiation source to position a radiation source offset with regard to the longitudinal axis to form an asymmetric radiation dosing profile.

In another embodiment, a brachytherapy treatment device includes at least one tubular insertion member and an expandable member. The at least one tubular insertion member having a proximal end, a distal end, and a radiation source lumen disposed along longitudinal axis. The expandable member is disposed on and surrounds the distal end of the at least one tubular insertion member. The distal end of the at least one tubular insertion member within the expandable member has proximal and distal tip segments. The proximal and distal tip segments are in detachable mated engagement. Detaching the proximal and distal tip segments exposes the radiation source lumen of the at least one tubular insertion member to an interior volume of the expandable member, wherein the radiation source lumen is adapted to receive and position a radiation source within the interior volume of the expandable member to form an asymmetric radiation dosing profile.

In yet another embodiment, a brachytherapy treatment device includes an insertion support structure, at least one tubular member, and an expandable member. The insertion support structure has proximal and distal ends. The at least one tubular member has proximal and distal ends and is sized to be received by the insertion support structure. The at least one tubular member also has a radiation source lumen extending along a longitudinal axis. The expandable member defines an internal volume and is disposed on and surrounds the distal end of the at least one tubular member. The at least one tubular member is adapted to be independently positionable with regard to the insertion support structure. The at least one tubular insertion member is deflectable within the internal volume to expose the radiation source lumen to the internal volume to form an asymmetric radiation dosing profile.

In yet another embodiment, a radiation treatment device comprises a tubular member, an expandable device, a radiation source position, and an adjustable radiation source position mechanism. The tubular member has a longitudinal axis, a distal portion adapted to be inserted within a patient to a treatment site and a proximal portion adapted to extend out of the patient. An expandable device is disposed on the distal portion of the tubular member and is configured to be expanded such that tissue confirms to an outer surface, whereby such confirming tissue defines an inner boundary of target tissue to be treated by radiation. The radiation source position is located within said tubular member at a position axially corresponding to said first and second expandable devices. The adjustable radiation source position mechanism controllably adjusts the position of the radiation source position.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIG. 1 is a side view/schematic view of an exemplary brachytherapy applicator catheter;

FIG. 2 is a side, sectional, schematic view of the distal portion of an applicator and transfer catheter having an exemplary radiation shield;

FIG. 3 is a perspective schematic view of distal portion of a transfer catheter having another exemplary radiation shield;

FIGS. 4a-4c are side, sectional, schematic views of the distal portion of an applicator having multiple co-terminal balloons;

FIG. 5 is a side, sectional, schematic view of the distal portion of an applicator having multiple balloons;

FIG. 6 is a side, sectional, schematic view of the distal portion of an applicator having multiple balloons;

FIG. 7 is a perspective, schematic view of the distal portion of another applicator having multiple balloons:

FIG. 8 is a perspective, schematic view of the distal portion of an applicator having a segmented balloon;

FIG. 9 is side, sectional, schematic view of the distal portion of an applicator having an eccentrically shaped balloon;

FIG. 10 is side, sectional, schematic view of the distal portion of an applicator having a mechanical structure for modifying the shape of the balloon;

FIGS. 11a-11d are side, sectional, schematic views of a laminated, hybrid balloon for use on an applicator,

FIGS. 12a-12d are side, sectional, schematic views of another laminated, hybrid balloon for use on an applicator;

FIG. 13 is a side, partial-sectional, schematic view of an applicator having pull wires to adjust the location of a radiation source position;

FIGS. 14a and 14b are side views of intertwined helical tubes which may be utilized on an applicator to adjust the position of a radiation source position;

FIG. 15a is a partial-sectional, schematic view of an applicator having pre-stressed tube to adjust the location of a radiation source position;

FIG. 15b is a partial top view of the applicator of FIG. 15 showing the indexing feature of the device;

FIG. 16A illustrates a side view of an exemplary brachytherapy treatment device having a deflectable tubular insertion member and positioned at a treatment site;

FIG. 16B illustrates across-sectional view of FIG. 16A;

FIGS. 17A and 17B illustrate cross-sectional views of exemplary brachytherapy treatment devices having a deflectable tubular insertion member having differential wall thickness;

FIG. 17C illustrates a side view of an exemplary brachytherapy treatment device having a deflectable tubular insertion member formed of varying materials;

FIGS. 18A and 18B illustrate side views of exemplary brachytherapy treatment devices having a helical-shaped tubular insertion member;

FIG. 19 illustrates a perspective view of an exemplary brachytherapy treatment device having at least one tubular insertion member and a threaded member;

FIG. 20A illustrates a side view of an exemplary brachytherapy treatment device having first and second expandable members;

FIG. 20B illustrates a side view of an exemplary brachytherapy treatment device having first and second expandable members with the second expandable member inflated;

FIG. 20C illustrates a side view of another exemplary brachytherapy treatment device having first and second expandable members with the second expandable member inflated;

FIG. 21A illustrates a side view of an exemplary brachytherapy treatment device having an insertion support structure and independently moveable tubular member;

FIG. 21B illustrates a cross-sectional view of FIG. 21A;

FIG. 22A illustrates a side view of an exemplary brachytherapy treatment device having an insertion support structure and two independently moveable tubular members;

FIG. 22B illustrates a cross-sectional view of FIG. 22A;

FIG. 22C illustrates a cross-sectional view of an exemplary brachytherapy treatment device having an insertion support structure and four independently moveable tubular members; and

FIGS. 23A-23D illustrate side views of an exemplary brachytherapy treatment device having detachably mated proximal and distal tip segments.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments. The features illustrated or described in connection with one exemplary embodiment may be combined with the features of other embodiments. Such modifications and variations are intended to be included within the scope of this disclosure.

Disclosed herein are devices and methods for use in treating tissue disorders by the application of radiation, energy, or other therapeutic rays. While the devices and methods disclosed herein are particularly useful in treating various cancers and luminal strictures, a person skilled in the art will appreciate that the methods and devices disclosed herein can have a variety of configurations, and they can be adapted for use in a variety of medical procedures requiring treatment using sources of radioactive or other therapeutic energy. These sources can be radiation sources such as radio-isotopes, or man-made radiation sources such as x-ray generators. The source of therapeutic energy can also include sources of thermal, radio frequency, ultrasonic, electromagnetic, and other types of energy.

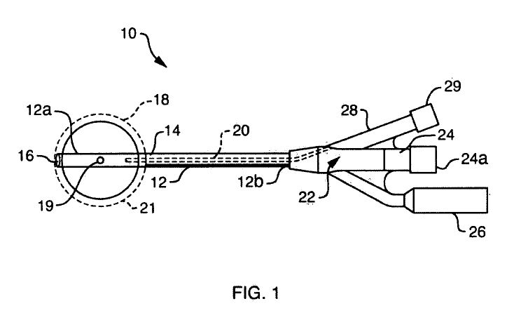

Referring first to FIG. 1, in general, a brachytherapy applicator 10 (also commonly referred to as an applicator catheter or treatment catheter) comprises an elongate tubular member 12 having a proximal end 12b, a distal end 12a, and a main lumen 14 extending therebetween. The distal end 12a is adapted to be inserted into the patient's body. The proximal end 12b is adapted to extend outside the patient's body. The walls of the tubular member 12 are substantially impermeable to fluids, except for any apertures and openings in the walls of the tubular member.

The main lumen 14 may be configured to receive a distal end of the transfer catheter. The main lumen 14 has an aperture 16 at, or near, its distal end that is in fluid communication with the exterior of the tubular member 12. The aperture 16 may simply be an open end of the tubular member 12, or it can be an opening in the wall of the tubular member 12. The aperture 16 allows bodily fluids to enter the main lumen 12 when the applicator 10 is positioned in a patient's body. An expandable device 18, such as a balloon, is provided on the distal end 12a of the tubular member 12.

The expandable device 18 can be any device which can be controllably expanded and contracted to retract tissue, such as a balloon, a cage, or other device. An expansion link 20, such as a balloon inflation tube, is disposed within the main lumen 26 and extends from the expandable device 18 to the proximal end 24a of the tubular member 12. Depending on the form of the expandable device 18, the expansion link 20 could comprise a mechanical linkage, an electrical connection, or other suitable link for remotely expanding and contracting the expandable device 18. Alternatively, the expansion link 20 can be provided on the exterior of the tubular member 12, or it can be integrally formed with the tubular member 12. The expansion link 20 allows the expandable device 30 to be controllably expanded and contracted from a location at the proximal end 12b of the tubular member 12, such as by delivering an inflation fluid to a balloon through an inflation tube.

The distal portion 12a of the tubular member 12 is adapted to receive a radiation source (not shown) and to position the radiation source within the expandable device 18 at a radiation source position 19. A radiation source position at the radiation source position 19 will produce an exemplary isodose profile 21 relative to the surface of the expandable device 18.

A hub 22 is disposed on the proximal end l2b of the tubular member 24. The hub 22 has a plurality of ports 24, 26 and 28. The first port 24 has a first port lumen 24a which is in fluid communication with the main lumen 14. The first port lumen 24a is preferably axially aligned with the axis of the tubular member 12.

The hub 22 also has a second port 26 which is in fluid communication with the main lumen 14 such that fluid can be drained through the aperture 16 located at a treatment site within a patient's body, through the main lumen 26 and out of the patient's body. To collect the fluid, the second port 26 may be configured to connect to a fluid drainage bag, such as a urine drainage bag.

The hub 22 also includes a third port 28 which is coupled to the expansion link 20. In the case that the expandable device 18 is a balloon, the third port 28 has a lumen in fluid communication with the inflation lumen 20. The third port 28 may have an interface 29 which is configured to be coupled to a source of inflation fluid, such as a hose or a syringe.

The hub 22 may be formed in any suitable fashion as known by those skilled in the art. For example, the hub 22 may be integrally formed of plastic or other suitable material. Moreover, the hub 22 may include additional ports, as needed for the particular application of the applicator 10. For instance, the applicator 10 could have more than one balloon, as described below for many of the devices, wherein each of the balloons is independently inflatable. Thus, the hub 22 could have an additional port for each additional balloon.

Indeed, the applicator 10 may have all of the features and aspects of the applicator catheter described in co-pending U.S. Patent Application 60/870,690, entitled “Brachytherapy Device and Method,” filed on Dec. 19, 2006, and in copending U.S. Patent Application entitled “Selectable Multi-Lumen Brachytherapy Devices and Methods,” filed on or about Dec. 18, 2007, each of which is incorporated by reference in their entirety herein.

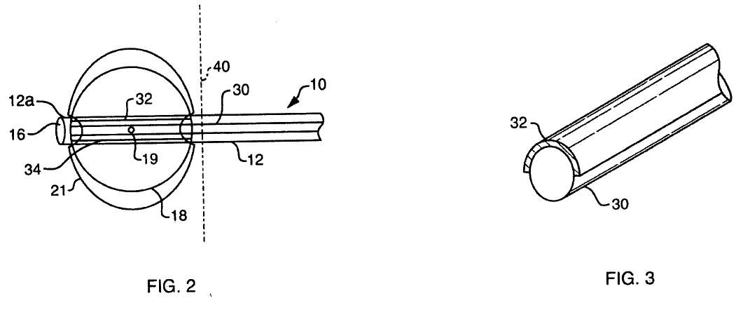

Turning now to FIG. 2, in a first exemplary embodiment for producing an asymmetric radiation dosing profile, an applicator 10 is utilized in conjunction with a transfer catheter 30 having a tube 31 and a radiation shield 32. FIG. 2 is a schematic view which shows only the distal portion 12a of the applicator 10 and the distal portion of the transfer catheter 30, but it is understood that the applicator 10 may include any of the features of the applicator 10 as described above, and any of the features of transfer catheters as described and referenced herein. The radiation shield 32 is disposed on a distal portion 34 of the transfer catheter 30 which is positioned at the radiation source position 19 when the transfer catheter 30 is installed in the applicator 10. The radiation dosing profile 21 shows that the shield 32 re-shapes the radiation dosing profile so that it is elliptical in cross-section, rather, as opposed to the circular shape without the shield 21 (see FIG. 1).

The radiation shield 32 comprises an elongate cylinder having an annular cross-section which may extend the entire diameter of the expandable device 18, or any other desired length to provide the desired radiation dosing profile. The shield 32 may be formed of a metallic material or any other material which attenuates radiation. The annular shield 32 creates an asymmetric radiation profile because the radiation is more attenuated in the directions in which the radiation path through the shield is great, i.e. in oblique directions through the shield 32, whereas the minimum attenuation occurs in the direction perpendicular through the shield 32 which has the shortest path through the shield 32.

This configuration of shield 32 can be used advantageously by placing the device with the axis of the tubular member 12 perpendicular to sensitive tissue 40, such as skin. Because of the attenuation in the axial direction, the minimum distance between the balloon 18 and the sensitive tissue 40 can be reduced. As a result, brachytherapy can be performed in a position that may not have been possible due to exposing the sensitive tissue 40 to unsafe radiation levels. The shield 32 can also be used to direct and shape the radiation profile to minimize unnecessary exposure to healthy tissue.

Because the shield 32 is removable with the transfer catheter 30, the shield 32 can be removed so that it does not interfere with pre-radiation treatment imaging or other procedures, and installed during the radiation treatment.

The shield 32 can be configured with different shapes and sizes in order to provide particular radiation dosing patterns. For example, in the embodiment of FIG. 2, the shield 32 extends the length of the balloon, while in other implementations, the shield 32 can extend for only a portion of the balloon diameter or extend beyond the balloon diameter. For instance, the shield 32 can cover only the half of the diameter of the balloon closest to the sensitive tissue 40, in order to attenuate radiation dosing primarily in the direction of the sensitive tissue 40, but not in directions distal to the shield 32. In another example, the shield can be a half-cylinder as shown in FIG. 3, such that the shield 32 only attenuates radiation over half the balloon 18, such as in the direction of sensitive tissue.

The shield can also include one or more apertures of varying sizes and diameters in order to provide a particular dosing pattern. For example, an aperture can be used to focus the radiation to a particular area of tissue while attenuating other areas.

The shield 32 can be composed of different materials or materials having different thickness in order to provide varying degrees of radiation attenuation. For example, thick or denser materials can be used to provide greater attenuation, which can be localized or directed to produce a desired dosing pattern. Additionally, the shield can be a composite of more than one material or thickness in order to provide varying degrees of attenuation. For example, the shield can be thick in the direction of sensitive tissue in order to reduce or even block the radiation dose applied to the sensitive tissue, and/or the shield can be thinner in the opposite direction in order to provide a higher radiation dose to the target tissue.

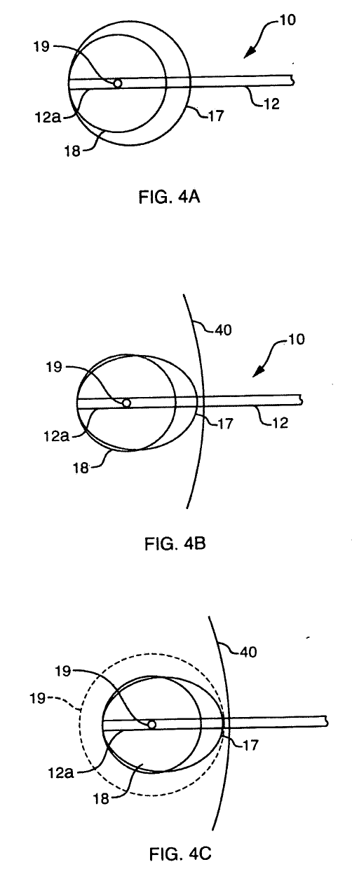

Turning now to FIGS. 4-8, various applicators 10 having multiple expandable devices (e.g. balloons) for shaping the tissue to provide various dosing patterns are illustrated. In contrast to the radiation shields just described which shape or direct the radiation pattern, these devices are designed to move tissue in relation to the radiation dosing profile to adjust the levels of radiation exposed to different areas of tissue. Each of the balloons, or balloon segments, can have separate inflation lumens so that each balloon can be independently inflated to create a desired tissue configuration about the radiation source position.

For example, in FIG. 4, the applicator 10 has an inner balloon 18 which is mounted co-terminally (i.e. one side of each balloon is positioned at the same point, in this case, the distal end of the tubular member 12) with an outer balloon 17. Separate inflation lumens (not shown) are provided for independent inflation of the balloons, 17 and 18. The inner balloon 18 has a smaller inflated diameter than the outer balloon 17.

In use, the inner balloon is inflated, for example, to fill a lumpectomy cavity. Thus, the surrounding tissue substantially conforms to the outer surface of the inner balloon 18. Then, the outer balloon 17 is inflated (using lower pressure), which asymmetrically increases the distance between the inner balloon 18 and the tissue on the proximal side of the inner balloon 18. As shown in 4b, the outer balloon 17 only inflates in a “bubble” in the region not tightly distended by the inner balloon 18, such as the area beneath the skin. Since the radiation dose from a source at the radiation source position 19 decreases by the square of the distance, the radiation dose received by the skin 40 is reduced to a safe level, thereby allowing brachytherapy treatment in this area. The use of two balloons does not have to change the standard calculated treatment planning target volume (“PTV”) because the two balloons can be constructed to be radiographically distinct, with the inner balloon 18 being denser than the outer balloon 17, or both balloons can be substantially transparent to the radiation, or the outer balloon can be substantially transparent to the radiation. In any of these cases, the PTV 21 can be based on the spherical inner balloon 18 as shown in FIG. 3. As clearly illustrated in FIG. 3, the outer balloon 17 has distended the skin 40 to a position outside of the PTV 21. Thus, skin 40 which would have previously been within the PTV, preventing standard treatment, is now outside the PTV 21 due to the displacement caused by the outer balloon 17. Moreover, since both balloons can be inflated independently, the single device of FIG. 4 can be used to provide a broad range of conventional spherical treatment volumes depending on which balloon is inflated and the degree of inflation.

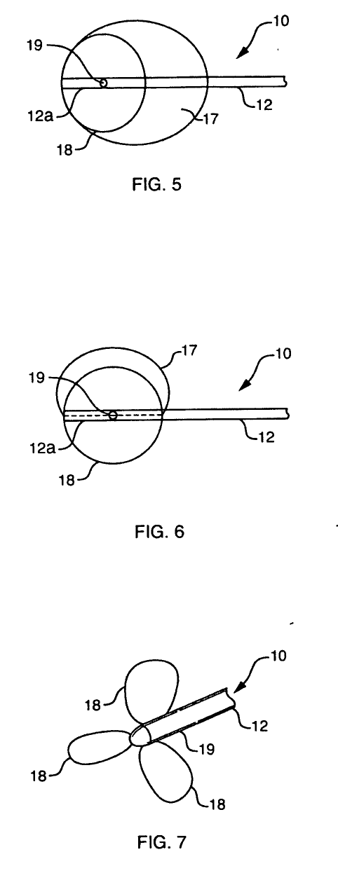

Moreover, modifications to the shape of the inner balloon 17 and outer balloon 18 can allow a wide variety of shapes for particular applications. For example, FIG. 5 shows another exemplary multiple balloon applicator 10 in which the tail spacing on the inner balloon 18 is shortened and the tail spacing of the outer balloon is lengthened. Consequently, the inner balloon 18 takes on an oblate shape upon inflation and the outer balloon 17 becomes more elliptical. A similar result is achieved which is to increase the distance along the axis parallel to the direction of insertion (the axis of the tubular member 12) to reduce the radiation dose received by tissue located in that direction.

The multiple balloon applicators may also include multiple balloons where some or all of the balloons are not within other balloons. One example is shown in FIG. 6. In this embodiment, a second balloon 17 having a hemispherical shape is mounted over one side of the inner balloon 18. Filling the second balloon 17 offsets the tissue in a direction transverse to the axis of the tubular member 12, as opposed to the devices above which move the tissue substantially in a direction parallel to the axis of the tubular member 12.

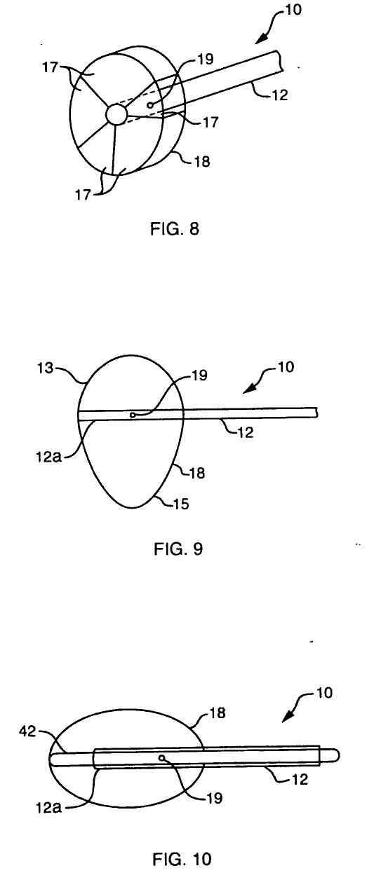

Additional exemplary embodiments of balloon applicators are shown in FIGS. 7 and 8. The applicator 10 of FIG. 7 has three lobed balloons 18 which extend from the tubular member 12 in different directions. Selective inflation of each balloon 18 can provide for many different patterns of the target tissue in order to provide the desired radiation dosing profile. Any number of lobed balloons 18 can be used, for example, 2, 3, 4, 5, 6 or more are possible. The applicator 10 of FIG. 8 has a balloon 18 which is divided into five independently inflatable segments 17. Similar to the lobed balloons of the applicator 10 of FIG. 7, each of the balloon segments 17 can be selectively inflated to provide for many different patterns of the target tissue in order to provide the desired radiation dosing profile.

In order to further adjust the radiation dosing profile when using any of the balloon devices, contrast fluid can be used to inflate any one or more of the balloons on a device. In this way, the balloon itself acts a shield which attenuates radiation tending to transmit through the balloon. For instance, the outer balloon 17 on the applicator 10 of FIG. 4 can be filled with a radiation attenuating fluid such that the balloon 17 also acts as a radiation shield for the sensitive tissue 40. Different balloons can be filled with contrast fluids having differing radiation shielding properties to provide even more radiation dosing profile possibilities. Additionally, the use of different contrast agents in the balloons can be used for imaging purposes to determine the position of each balloon.

A single balloon on an applicator can also provide for the shaping of target tissue in order to provide an asymmetric dosing profile. For instance, the balloon can simply have an eccentric shape, such as the balloon 18 shown in FIG. 9. The balloon 18 of FIG. 9 has an eccentric protrusion 15 and a spherical portion 13. The applicator 10 of FIG. 9 need only be oriented with the eccentric protrusion in the direction of the sensitive tissue. An eccentric shape can also be obtained by constructing the balloon 18 from materials that provide differential expansion (i.e. different portions of the balloon expand at different rates during inflation). In one implementation, different material thicknesses can be used to provide differential expansion. Alternatively, the balloon can be a composite of different materials having different mechanical properties. For example, the eccentric protrusion 15 can be formed of a more flexible material, while the spherical portion is formed of a stiffer material.

Alternatively, in another embodiment, a mechanical structure can be used to modify the shape of the balloon. For instance, a mechanism can be used to elongate one or both ends of an otherwise spherical balloon. A ratcheting or screw mechanism could be used to incrementally distend the distal end of the balloon to a desired shape.

One exemplary embodiment of such a device is shown in FIG. 10. A single rigid shaft 42 is used to elongate the balloon 18 by applying a force to the distal end of the balloon 18. The rigid shaft 42 can have a proximal end that extends through the tubular member and out of the patient so that it can be manipulated.

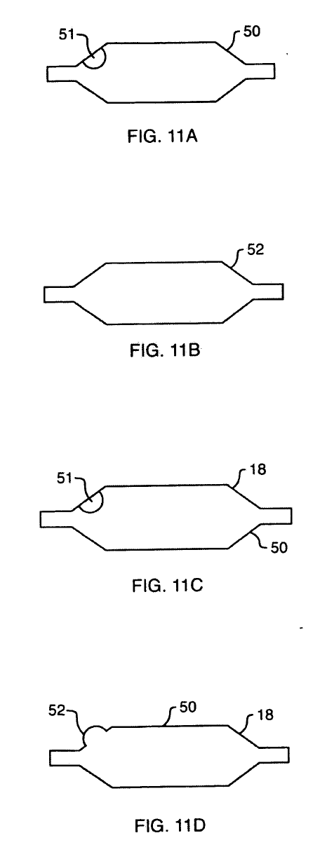

Referring now to FIGS. 11a-11d, another balloon 18 for use on an applicator 10 comprises a hybrid balloon having a relatively non-compliant outer balloon or sheath 50 and a compliant inner balloon 52. As shown in FIG. 11a, the outer balloon or sheath 50 has any desired shape for the particular application, and one or more openings 51, which can be shaped and located to provide the desired shape when the inner balloon 52 protrudes through the opening(s) 51. The inner balloon 52 has a shape that is similar to the shape of the outer balloon 50 as shown in FIG. 11b. The inner balloon 52 and outer balloon 50 are laminated together to form the assembled structure as shown in FIG. 11c. When the inner balloon is inflated to a first pressure, it will expand the outer balloon 50 until the outer balloon 50 is fully open to its non-compliant shape, as shown in FIG. 11c. At that point, further inflation of the inner balloon 52 will cause the compliant inner balloon 52 to expand out of the opening 51 thereby displacing the tissue surrounding this area further from the radiation source position.

The inner balloon 52 may be formed, for example, of such compliant materials such as silicone, polyurethane, and low durometer thermal plastic elastomers such as Pebax and Hytrel. Non-limiting examples of non-compliant materials for forming the outer balloon 50 include polyethylene, PET, nylon, and high durometer thermal plastic elastomers such as Pebax and Hytrel. One example includes an inner balloon 52 molded from a low durometer Pebax (25 D) and an outer balloon 50 formed from a higher durometer Pebax (72 D). The inner balloon 52 can be bonded (laminated) to the outer balloon 50 using any suitable techniques known to those of skill in the art. Bonding techniques include, without limitation, polymer bonding (where using a chemical reaction, polymer bonds are broken and reformed, UV curable bonding (a bonding agent is applied to the balloon surfaces and cross linked by a UV source applied to the surface), heat bonding (the outer balloon is placed over the inner balloon and compressed/suctioned against a heated mandrel, or heat is radiated externally with the balloons pressurized to force the two surfaces against a mold, thus bonding the two balloons, or laser bonding (where a laser source is used to excite the polymer bonds and laminate the two balloons at critical points).

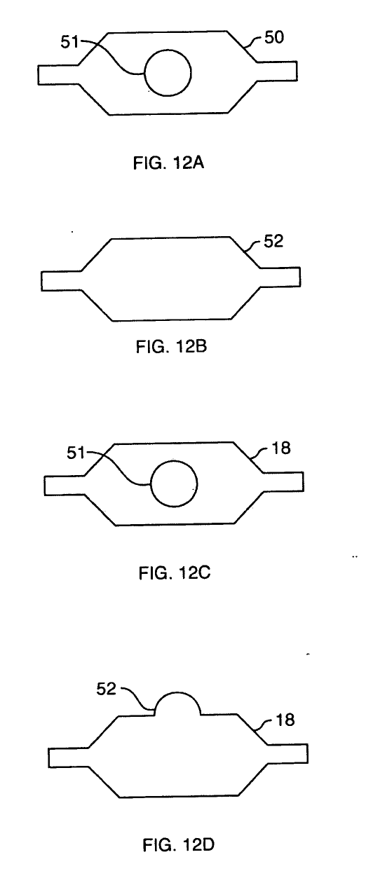

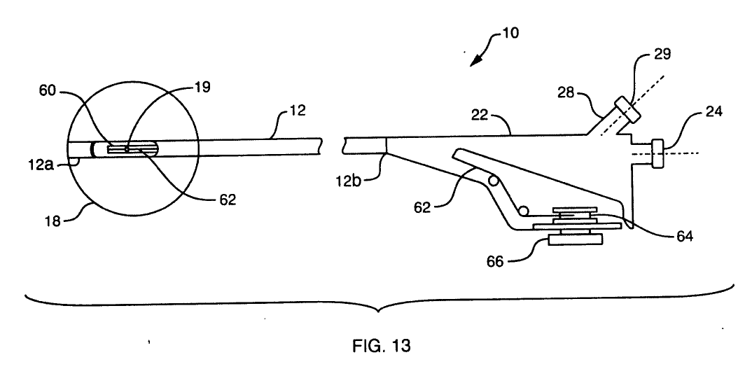

FIGS. 12a -12d illustrate another hybrid balloon 18 which is identical to the hybrid balloon of FIGS. 11a-11d, except that the opening 51 in the outer balloon 50 has a different configuration (e.g. a different location). Accordingly, it can be seen that the hybrid balloon can have any desired shape and opening(s) configured to provide the desired displacement of tissue to obtain a particular asymmetric radiation dosing profile.

Although balloons are included in many of the described embodiments herein, the balloons, such as the balloons 18 shown in the figure and described herein, may also comprise a basket catheter formed of a shape memory alloy such as nitinol or shape memory plastic. The outer surface of the expandable device 18 may then be distorted using either pull wires located within the tubular tines of the catheter or by displacing the proximal end of the basket catheter tines relative to each other. The distorted outer surface results in an asymmetric isodose profile in the target tissue surrounding the expandable device.

Turning now to FIGS. 13-15, various devices for implementation on an applicator which can adjust the position of the radiation source position relative to the expandable device(s), or other reference point on the applicator, will now be described. These devices allow the displacement of the location of the radiation source position 19 with respect to the position of the balloon 18 to achieve an asymmetric radiation dosing profile. By orienting the azimuth of the offset plane with respect to the resected volume, a physician may select an isodose profile that provides the desire therapeutic effect to the target tissue, while reducing the radiation dose received by sensitive tissue.

The applicator 10 of FIG. 13 utilizes a flexible tube or shaft 60 near the distal end 12a of the tubular member 12. The flexible tube 60 is operably coupled to a set of pull wires 62 (usually 2 or more), similar to the structure used to control the instrument tip on some endoscopes. The proximal end of the pull wires 62 are coupled to pulley 64 which is controlled by a thumbwheel 66. Thus, the radiation source position 19 may be moved radially within the control space defined by the balloon 18 via the pull wires 62. A locking feature, such as a detent or ratchet, may be provided on the thumbwheel to maintain the position of the flexible tube 60 in a set position. In addition, the pull wire system could include a feedback control system to maintain the flexible tube in a set position relative to some reference frame, such as the radial distance from the radiation source position 19.

Alternative to the use of pull wires 62, the applicator 10 could be configured to use an electric field to move the flexible tube 60 and consequently the radiation source position 19. A controllable electric field module is placed at the position of the flexible tube 60. A feedback control system is operably coupled to the electric field module. The position of the radiation source position can then be controlled with the control system by modifying the electric field to move the flexible tube 60.

In still another implementation of the applicator as shown in FIG. 13, the pull wires 62 and flexible tube 60 can be replaced with inner and outer concentric pre-bent tubes. The tubes may be formed of nitinol or other suitable material. When the bends are oriented in the same azimuth, the maximum offset of the radiation source position is achieved. When the bends are oriented 180 degrees apart, the tubes will tend to straighten each other, resulting in little or no offset from the axis of the tubular member 12. The bends in the concentric tube assembly can be aligned in the relative orientation to achieve the offset which results in the desired radiation dosing profile in the target tissue.

In yet another implementation of the applicator as shown in FIG. 13, instead of using the pull wires 62 to move flexible tube 60, a magnetic field can be provided by magnets located around the target tissue. The flexible tube 60 is magnetized such that the magnets move the flexible tube 60 to achieve the desired offset of the radiation source position and hence the desired radiation dosing profile.

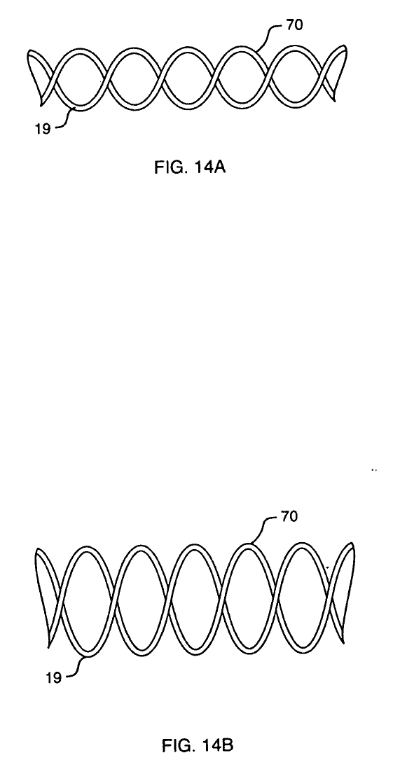

In another alternative embodiment, the pull wires 62 and flexible tube 60 can be replaced by a pair of intertwined helical tubes 70 as shown in FIGS. 14a and 14b. When the ends of the tubes 70 are extended away from each other, the radiation source position is close to the axis of the tubular member 12, as shown in FIG. 14a. When the ends of the tubes 70 are compressed, the tubes move outward relative to the helix axis, thereby moving the radiation source point further away from the axis. When the desired offset is achieved, the radiation source is introduced into one of the offset tubes and is inserted to the radiation source position 19. The entire intertwined tube assembly may be rotatable in order to orient the offset in the desired direction to achieve the desired radiation dosing profile in the target tissue.

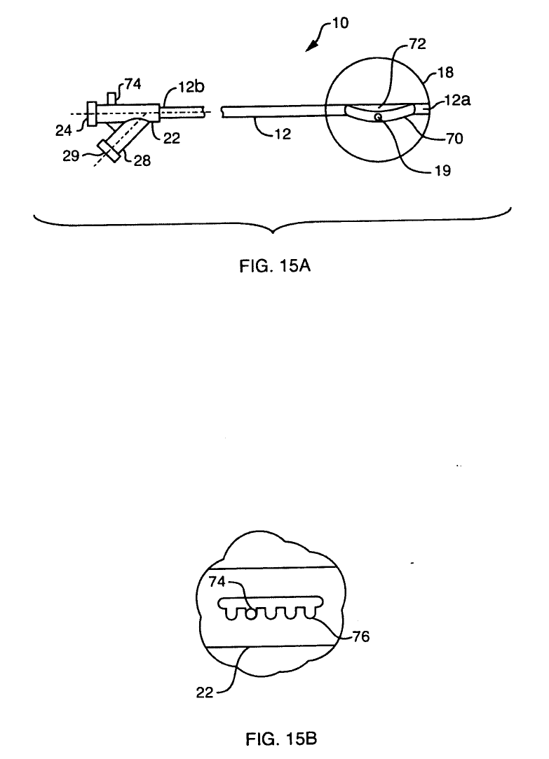

FIG. 15 shows one more exemplary embodiment of an applicator 10 having a mechanism for offsetting the location of the radiation source position 19 relative to the axis of the tubular member 12. The applicator 10 further comprises a flexible pre-bent tube 70 and a window 72 cut into the tubular member 12. The proximal end of the pre-bent tube 70 is coupled to a locking pin 74 which removably couples to a series of detents 76. While the bend in the flexible tube 70 is mostly spaced away from the window 72, the bend in the tube is straightened and the radiation source position 19 is nearest the axis of the tubular member 12. As the bend is progressively moved into the window 72, the tube 70 takes on its bent shape by protruding out of the window 70, thereby moving the radiation source position 19 away from the axis of the tubular member 12. When the desired offset is achieved, the locking pin 74 is secured in the applicable detent 76 to lock the tube 70 and radiation source position 19 in a set position.

Methods for delivering radioactive treatment to a patient are also provided herein. The method for performing brachytherapy using the devices described herein may be as described in co-pending U.S. Patent Application 60/870,690, entitled “Brachytherapy Device and Method,” filed on Dec. 19, 2006. Therefore, the methods disclosed herein will only be described generally. The method typically begins with placing the applicator catheter 10 within the patient. Prior to this step, it is common for a surgery to have been performed to remove as much of a tumor as possible. A surgical resection of the tumor is typically performed, thereby leaving a surgical pathway and resected space for placement of the applicator catheter 10 within the patient. In certain embodiments, the step of placing the applicator catheter 10 includes surgically resecting, incising or otherwise altering a patient's tissue.

The applicator catheter 10 is inserted into the patient, with the expandable device 18 in a contracted configuration such that the distal end 12a is positioned at or near the treatment site, i.e. the site of the surgical resection of the tumor. The proximal end 12b of the applicator 10, including the hub 22, extends outward from the patient.

The expandable device 18 is expanded through use of the expansion link 20 to create the desired distance between the tissue and the radiation source position 19. The respective radiation dose shaping devices and tissue shaping devices for the particular applicator 10 being utilized is operated to achieve the desired radiation dosing profile in the target tissue. An afterloader operates to deliver a radiation source to the radiation source position so as to dwell within the applicator 10 for a desired or prescribed period of time. Once the treatment time is complete, the afterloader retracts the radiation source out of the applicator 10. The expandable device 18 may then be returned to its contracted configuration (e.g. deflating a balloon).

The applicator 10 may remain within the patient's body in the treatment position so that it can be used at the next treatment session, or it can be removed.

One skilled in the art will appreciate further features and advantages of the devices and methods disclosed herein based on the above-described embodiments. Accordingly, these devices and methods are not to be limited by what has been particularly shown and described, except as indicated by the appended claims. All publications and references cited herein are expressly incorporated herein by reference in their entirety.

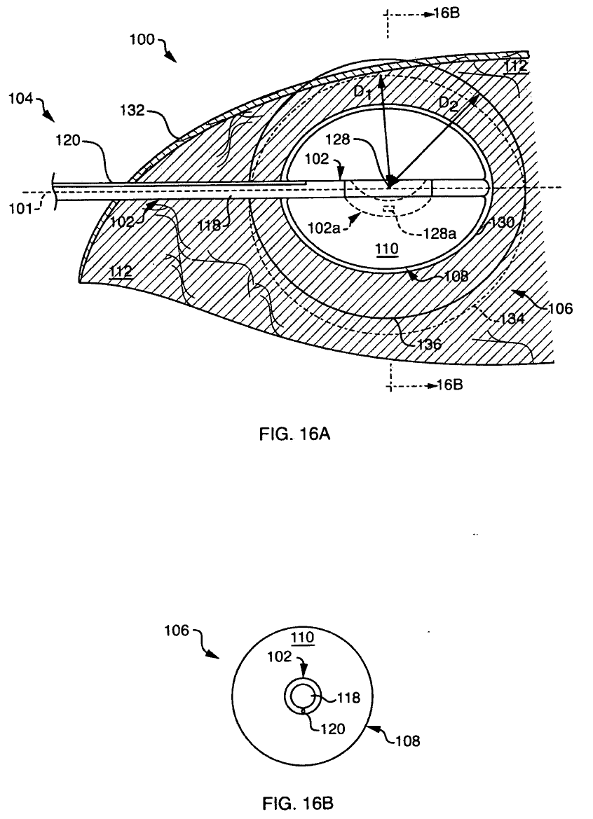

The brachytherapy treatment devices and methods disclosed herein provide a radiation dosing profile which may be oriented in any number of configurations. In some embodiments the radiation dosing profile generated may be asymmetrical to protect sensitive tissues while still allowing target tissues to receive an appropriate therapeutic dose of radiation. Referring now to FIGS. 16-23, like numerals indicate like features throughout the drawing figures shown and described herein.

FIG. 16A illustrates a first embodiment of a brachytherapy treatment device 100 having a tubular insertion member 102 which may be deflected, bent, articulated, or otherwise distorted (exemplary deflection shown also as 102a in dashed lines) to create an asymmetric dosing profile to protect sensitive tissues 132 while still allowing target tissues 112 to receive an appropriate therapeutic dose of radiation. A brachytherapy applicator or treatment device 100 (also commonly referred to as an applicator catheter or treatment catheter) may comprise at least one elongated tubular insertion member 102 having a longitudinal axis 101 extending its length between a proximal end 104 and a distal end 106. The distal end 106 of the tubular insertion member 102 is adapted to be inserted into a patient's body and the proximal end is adapted to extend outside of the patient's body. The tubular insertion member 102 should be rigid enough to provide an easy insertion profile for a surgeon, while still being soft and flexible enough to be comfortable for a patient during treatment. In some embodiments, a device 100 may include a plurality of tubular insertion members 102.

The tubular insertion member 102 may be formed of a flexible material, including without limitation various plastic or elastomeric polymers and/or other suitable materials. The tubular insertion member 102 should be flexible and soft enough that it conforms to surrounding tissue 112 and easily bends when force is applied, such as by movement of the patient's body (shown in part as tissue 112), making the tubular insertion member 102 more comfortable. The tubular insertion member 102 may further comprise a malleable element (not shown) adapted to confer a shape upon at least a portion of its length. The walls of the tubular insertion member 102 may be substantially impermeable to fluids, except where there are apertures and/or openings disposed within the walls of the tubular insertion member 102.

As shown in FIGS. 16A and 16B, the device 100 may further comprise an expandable member 18 disposed on and surrounding the distal end 106 of the at least one tubular insertion member 102 and having an inner surface defining a three-dimensional volume 110. The volume 110 defined by the expandable member 108, when inflated, should be substantially similar to the volume of the cavity 130 to substantially fill the cavity 130 and help provide a substantially uniform and symmetrical boundary. The expandable member 108 may be any device which can be controllably expanded and contracted to retract surrounding tissue 112, such as a balloon, bladder, or other device. The expandable member 108, when inflated, provides spacing between the at least one tubular insertion members 102 and the surrounding tissue 112.

The expandable member 108 may be formed of a variety of different materials. such as biocompatible polymers. Some exemplary biocompatible polymers may include silastic rubbers, polyurethanes, polyethylene, polypropylene, and polyester, just to name a few examples. The walls of the expandable member 108 will be formed of a radiation transparent material to allow radiation to pass through the walls of the expandable member 108 to treat the tissue 112 of the cavity 130 surrounding the expandable member 108. In some embodiments, it may be desirable to use one or more expandable members 108 or a double-walled member to minimize the risk of fluid leakage from the expandable member 108 into a patient (shown as tissue 112), such as may occur if one expandable member 108 becomes punctured.

As shown in FIG. 16B, the at least one elongated tubular insertion member 102 may also include a main lumen 118 extending between and operably coupling the proximal 104 and distal 106 ends of the tubular insertion member 102. The main lumen 118 may be a radiation source pathway configured to receive a radiation source and provide a pathway for positioning a radiation source at radiation source position 128 within the expandable member 108. It should be understood that deflection of tubular insertion member 102 results in deflection of main lumen 118 (disposed within tubular insertion member 102) to deflect or alter the radiation source pathway. It should also be understood that the deflecting embodiments described herein refer to deflection of main lumen/radiation source pathway 118 to deflect the radiation source to create an asymmetric radiation dosing profile. In some implementations, it may be possible to deflect radiation source pathway or main lumen 118 without deflection of tubular insertion member 102.

In alternative embodiments, there may be multiple radiation source lumens configured to receive a radiation source and provide pathways for positioning a radiation source at similar or different positions within the expandable member 108. The main lumen 118 of the tubular insertion member 102 may further comprise a plurality of other tubes or lumens (such as 120) disposed therein to provide several separate and independently operable pathways for accessing the distal end 106 of the tubular insertion member 102 via the proximal end 104 of the tubular insertion member 102. The main lumen 118 may further comprise an inflation lumen 120, such as a balloon inflation tube 120, disposed within the main lumen 118 and fluidly coupling the expandable member 108 and the proximal end 104 of the tubular insertion member 102. The inflation lumen 120 provides a fluid pathway, allowing the expandable member 108 to be remotely expanded/inflated and contracted/deflated from a location at the proximal end 104 of the tubular insertion member 102, such as by a user or machine. In some embodiments, the main lumen 118 may comprise multiple inflation lumens 120 for inflating multiple expandable members 108.

To deliver the brachytherapy treatment to a treatment site within a patient, the radiation source (not shown) may be placed at the radiation source position 128 (e.g., treatment site 112) via the tubular insertion member 102, as shown in FIG. 16A. Once placed at the treatment site 112, the radiation source creates a radiation dose distribution profile 136 which takes the shape of spherical isodose shells that are centered on the location of the radiation source. When the radiation source within the expandable member 108 is positioned close to sensitive tissue, such as skin 132, it is possible that the sensitive tissue 132 may receive an undesirably high radiation dose.

The issue of protecting sensitive tissues 132, such as skin, is commonly referred to as skin spacing, and is an important consideration in treatment planning. It may be necessary to ensure sufficient tissue depth exists (shown as D₁) between sensitive tissues 132 and the radiation source position 128 to prevent damage to the sensitive tissues 132 during treatment. Formation of an asymmetric dosing profile (shown as 134) by deflecting the tubular insertion member (deflection shown in dashed lines as 102a) provides a means for effectively treating areas where tissue depth (shown as D₁) is minimal between sensitive tissues 132 and the radiation source position 128.

The radiation dose profile 136 from a radiation source (positioned at radiation source position 128) is typically emitted substantially equally in all 360° surrounding the radiation source position 128, assuming the radiation source has no abnormalities. Thus, a radiation source positioned at the radiation source position 128 will emit radiation to produce an isodose profile 136 uniform relative to the inner boundary 130 of target tissue 112 to be treated. As shown in FIG. 16A sensitive tissue 312 (e.g., skin, bone, or other sensitive organs) falls within the radiation does profile 136 and thus may receive an undesirably high dose of radiation, resulting in damage to the skin 132.

With continuing reference to FIG. 16A, the distance (D₁) or spacing between the skin 132 and radiation source position 128 is substantially less than that of the distance (D₂) between the other surrounding target tissues 112 and that of the radiation source position 128. Because the radiation dose is emitted substantially equally in all directions. and because it decreases based upon the square of the distance, the proximity of the skin 132 to the radiation source 128 results in the skin 132 receiving an undesirably high and potentially very damaging dose of radiation. It is therefore advantageous to protect the skin 132 from receiving such a high dose of radiation by deflecting the tubular insertion member 102a (which repositions radiation source position 128a) to create asymmetric dose profile 134, which protects the skin 132 while still allowing the remainder of the target tissue 112 to receive a prescribed therapeutic dosage of radiation treatment.

An exemplary deflected tubular insertion member 102a is shown in dashed lines as 102a and an exemplary deflected radiation source position is shown as 128a (within the deflected tubular insertion member 102a). The deflection of tubular insertion member 102a reshapes the radiation dosing profile 136 into asymmetrical radiation dose profile 134 to enable an appropriate dose of brachytherapy treatment to be delivered, even when the treatment site is very close to sensitive tissues, such as skin 132. The deflection of the tubular insertion member 102a may be slight or more significant, but even a small deflection, such as 0.3 mm-1.5 mm, may have a significant impact upon the resulting isodose profile shape.

The deflected tubular insertion member 102a may also be used to direct, as well as reshape, the radiation dosing profile 134 to minimize unnecessary exposure to healthy tissue. The asymmetric radiation dosing profile 134 is shown in FIG. 16A as approximately circular, but may have a number of different configurations depending upon the particular radiation source used and the positioning, density, and/or radiation absorption properties of the tubular insertion member 102.

The distal end 106 of the at least one deflected tubular insertion member 102a (within the first expandable member 108) is offset from the longitudinal axis 101 of the tubular insertion member 102 when deflected, as shown in FIG. 16A. The at least one deflected tubular insertion member 102a is configured to receive a radiation source via main lumen 118 to position a radiation source off-center 128a of the longitudinal axis 101, forming an asymmetric radiation dosing profile 134. When multiple tubular insertion members 102 are utilized, they could all be deflected, bent, or articulated together in the same direction or they could be individually controlled in different directions.

The means for deflecting the at least one tubular insertion member 102 may include, but are not limited to: differential wall thicknesses; differential materials having differing durometer, column strength, or shape memory properties; pull-wires; threaded members such as turnbuckles or lead screws; a pre-stressed or pre-bent member; a second expandable member; a slide mechanism; a helical-shaped member; detachable proximal and distal tip segments; an insertion support structure adjacent to the tubular insertion member; and/or an adjustable radiation source position mechanism. The means for deflecting the at least one tubular insertion member 102 may include mechanisms operable to bend, kink, articulate, rotate, distend, buckle, or otherwise shape the at least one tubular insertion member 102 in a predictable and controlled manner. Each of these different deflection means will now be described in detail.

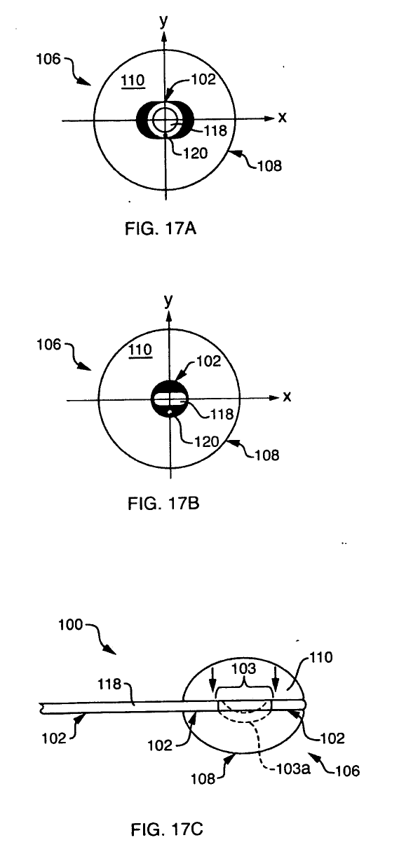

FIGS. 17A and 17B illustrate an exemplary brachytherapy treatment device 100 wherein the means for deflecting the at least one tubular insertion member 102 comprises a differential wall thickness. Varying wall thickness of the at least one tubular insertion member 102 causes member 102 to deflect in a predictable and known direction. This can be accomplished by a heterogeneous wall thickness utilizing the moment of inertia bias. In one embodiment, portions of the wall of the at least one tubular insertion member 102 have a width substantially thicker than that of remaining portions of the wall.

As shown in cross-section in FIG. 17A, the wall thickness in the horizontal or x-direction may be substantially thicker than the wall thickness in the vertical or y-direction. Conversely, as shown in FIG. 17B, the wall thickness in the y-direction may be substantially thicker than the wall thickness in the x-direction. If the wall thicknesses in the x-direction are thicker, the tubular insertion member 102 will deflect in the y-direction. Conversely, if the wall thicknesses in the y-direction are thicker, the tubular insertion member 102 will deflect in the x-direction. Wall thickness of the distal end 106 tubular insertion member 102 may be varied for a number of different lengths or sections of along longitudinal axis 101 within expandable member 108.

Wall thickness may be varied by utilizing a wall having an elliptical outer diameter with a concentric circular inner diameter (as shown in FIG. 17A), or an elliptical inner diameter with a concentric circular outer diameter (as shown in FIG. 17B). When tension or pressure is applied to the tubular insertion member 102, the tubular insertion member 102 will defect in the direction of the thinnest walls. In some embodiments, only a small portion of wall thickness may be varied to achieve desired deflection. In alternative embodiments, tubular insertion member 102 may have any number of different geometrical shapes and the wall thickness may be varied in any number of different geometrical shapes.

In an alternative embodiment shown in FIG. 17C, a portion of the at least one tubular insertion member 102 at the distal end 106 within the expandable member 108 may be formed of a different material 103. A portion 103 of tubular insertion member 102 may be formed of different materials or the same material having different properties, such as different thickness, strength, durometer, or column strength to provide deflection, as shown by dashed line portion 103a in FIG. 17C. In this implementation, the meeting points between differing materials (shown by arrows) between differing portion 103 and the remainder of tubular insertion member 102 may create a deflection point or fulcrum for deflecting the tubular insertion member (as shown in dashed lines at 103a). In this implementation, tubular insertion member 102 may be deflected by force exerted on distal end 106, proximal end 104, or a force or mechanism moving the distal 106 and proximal 104 ends toward one another.

With continuing reference to FIG. 17C, portion 103 of the at least one tubular insertion member 102 may be formed of a different material, such as shape memory material, for example. Shape memory polymers or alloys, such as nitinol may be used. In this embodiment, tubular insertion member 102 may be deflected by the internal or external activation or stimulation of the shape memory material. Shape memory materials may be utilized in conjunction with any of the embodiments described herein to achieve deflection of tubular insertion member 103 in controlled and predictable fashion.

Additionally, the at least one tubular insertion member 102 may be composed of different materials and/or combinations of materials having different properties in order to provide varying degrees of radiation absorption or attenuation. For example, thick or dense materials may be used to provide more attenuation, which can be localized or directed to produce a desired dosing pattern. Additionally, the at least one tubular insertion member 102 may be formed of a composite of more than one material or thickness in order to provide varying degrees of attenuation. For example, the at least one tubular insertion member 102 may be thicker in the direction of sensitive tissue 132 in order to reduce or even shield the radiation dose applied to the sensitive tissue 132 and/or the shield may be thinner in the opposite direction in order to provide a higher radiation dose to the target tissue 132.

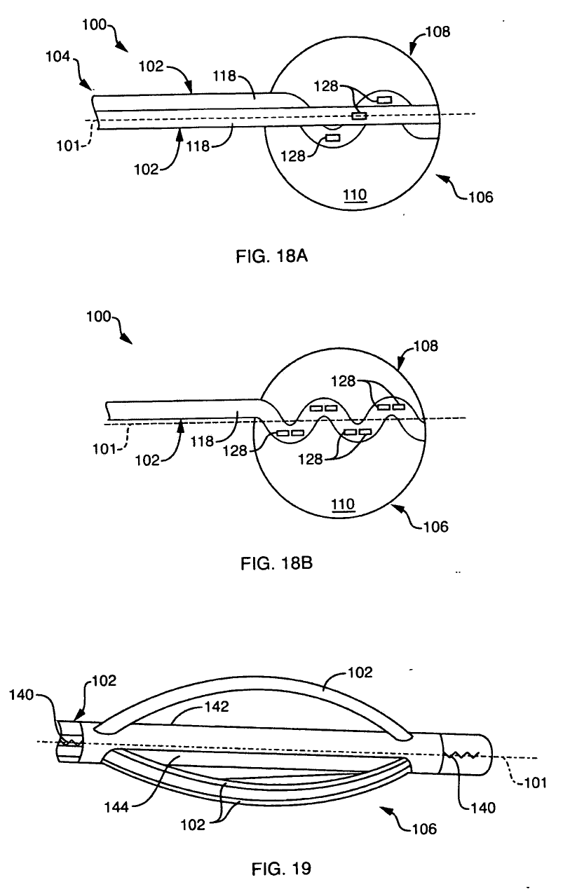

FIGS. 18A and 18B illustrate a brachytherapy treatment device 100 having at least one pre-stressed or curved tubular insertion member 102. The distal end 106 of the at least one tubular insertion member 102 (disposed within expandable member 108) may have a substantially curved or approximately helical-shape. The approximately helical-shape may be preformed or may be achieved after insertion of the device 100 into a patient, such as by removal of a cover or by stimulation to activate a shape memory material.

The helical-shaped tubular insertion member 102 is operable to receive and position a radiation source at a number of different radiation source positions (all shown as 128) along its length. The curved or helical-shaped tubular insertion member 102 may be formed in a swirl-like pattern providing a multitude of different radiation source positions offset from longitudinal axis 101 to provide a variety of different asymmetrical radiation dosing profiles. As shown in FIGS. 18A and 18B, the radiation source position 128 selected may be offset from the longitudinal axis 101 to form an asymmetric radiation dosing profile.

As shown in FIG. 18B, a plurality of tubular insertion members 102 may be utilized. A first tubular insertion member 102 may have a substantially straight configuration (disposed parallel to longitudinal axis of tubular insertion member 102) and a second tubular insertion member 102 may have a substantially helical-shaped structure. The helical shaped main lumen 118 provides a winding radiation source pathway providing a number of different varying radiation source positions 128 along its length to provide a wide variety of treatment planning options. While the tubular insertion member 102 is described herein as approximately helical, it may have any number of curved shapes, including a partially helical shape, such as a sin-wave shape.

FIG. 19 illustrates a perspective view of an exemplary brachytherapy treatment device 100 having a plurality of tubular insertion members 102 and a threaded member 140 operable to deflect the plurality of tubular insertion members 102. The threaded member 140 operably couples the proximal 104 and distal 106 ends of the device. The threaded member 140 may be operable to compress the distal end 106 of the tubular insertion member 102 within an expandable member 108 to deflect the plurality of tubular insertion members 102. The threaded member 140 provides a mechanism for precise, predictable and controlled deflection of tubular insertion members 102.

When a plurality of tubular insertion members 102 are utilized, a user may have the ability to select a particular one or a plurality of the tubular insertion members 102 for insertion of a radiation source, as disclosed in copending U.S. Patent Application filed on or about Dec. 18, 2007 and entitled “Brachytherapy Treatment Devices Having Selectable Lumens,” which is incorporated by reference herein for all that it discloses. The use of a plurality of tubular insertion members 102 and the ability to selectively choose one or more of those tubular insertion members 102 provides a user with a number of different asymmetric radiation dosing profiles providing a variety of different treatment planning options.

The threaded member 140 may comprise a number of different mechanisms. such as a turnbuckle or lead-screw design. A turnbuckle design may be applied by using a central pull-wire disposed such that relative motion can be achieved and tension shall be applied to the tubular insertion members 102. A known travel distance of the central pull-wire shall correlate to the distance and radius of deflection. The central pull-wire may have axial rigidity when tensioned to improve torque transfer of the turnbuckle design, but shall also be flexible in its relaxed state to provide patient comfort. The central pull-wire may also be hollow in order to allow for inflation of the expandable member 108 from the proximal end 104.

As shown in FIG. 19, the threaded member 140 may comprise a lead screw 140 operable to compress tubular insertion member 102 by moving the distal end 106 of device 100 slightly back toward proximal end 104. In this implementation a sheath 142 may be used to predictably deflect the one or more tubular insertion members 102. The sheath 142 may consist of multiple longitudinally disposed slits 144 that serve as openings or tracks through which the one or more deflected tubular insertion members 102 can protrude or deflect. FIG. 19 is shown without expandable member 108 for clarity of illustration herein only and it should be understood that this embodiment may also incorporate one or more expandable members 108 surrounding the distal end 106 of the one or more tubular insertion members 102.

In another embodiment, the means for deflecting the at least one tubular insertion member 102 may comprise a slide mechanism or a retractable sheath. The slide mechanism may be disposed on the proximal end 104 of the tubular insertion member 102 and may be operably coupled to the distal end 106. The slide mechanism may be operated via the proximal end 104 to slide toward the distal end 106 to compress the portion of the tubular insertion member 102 encompassed by the expandable member 108 to deflect the at least one tubular insertion member 102.

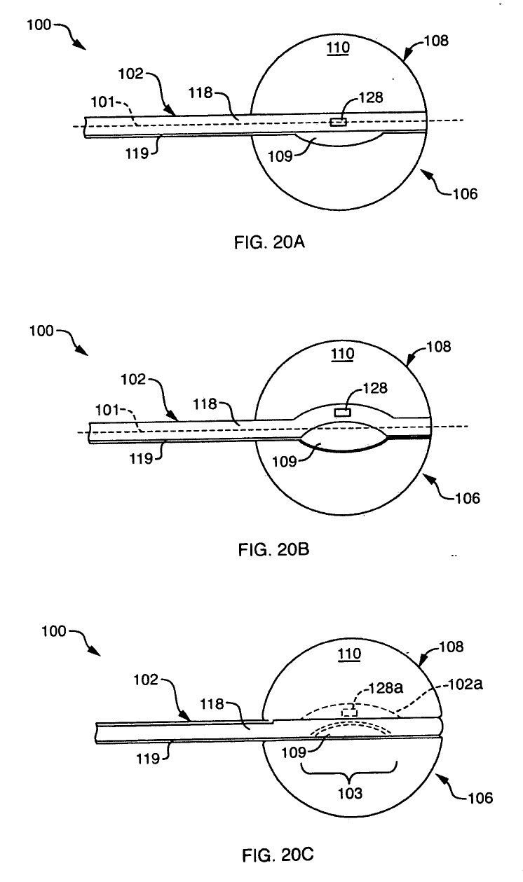

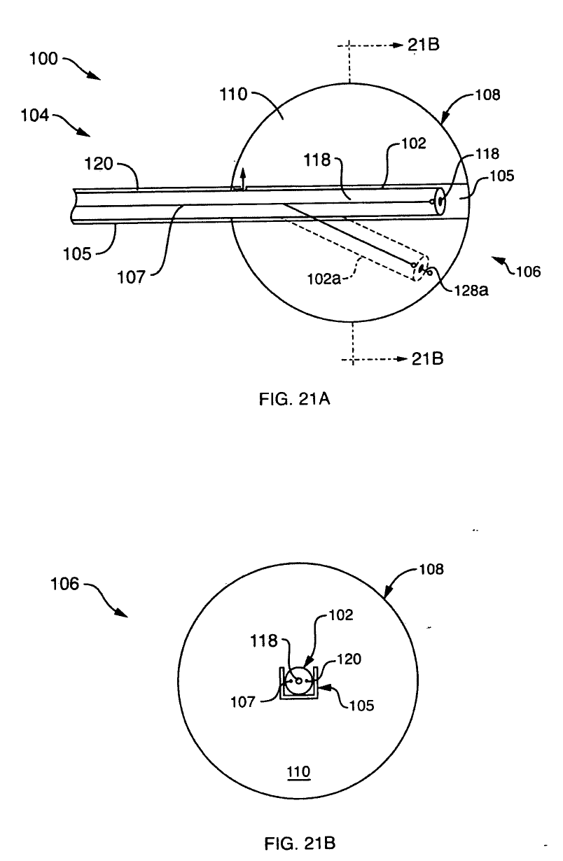

FIGS. 20A, 20B, and 20C illustrate another brachytherapy treatment device 100 having a means for deflecting the at least one tubular insertion member 102. As shown in FIGS. 20A-20C, the means for deflecting the tubular insertion member 102 may comprise a second expandable member 109 disposed adjacent the at least one tubular insertion member 102. Second expandable member 109 may be coupled to inflation lumen 119 and may be mounted coaxially or coterminally with first expandable member 108. FIG. 20A illustrates second expandable member 109 in a deflated or partially inflated state. FIG. 20B illustrates second expandable member 109 in an inflated state. Inflation of second expandable member 109 deflects the at least one tubular insertion member 102 to offset the at least one tubular insertion member 102 from the longitudinal axis 101 to offset radiation source position 128, as shown in FIG. 20B.

As also shown in FIGS. 20A and 20B, second expandable member 109 may have a thickened wall portion at a position furthest from the at least one tubular insertion member 102 to ensure expansion or inflation in a direction toward the at least one tubular insertion member 102. In other embodiments, either or both of the first and second expandable members 108, 109 may be molded to have an asymmetrical shape such that they have uniform wall thickness but inflate asymmetrically. As shown in FIGS. 20A and 20B, the second expandable member 109 may have a slightly rounded shape.

In another embodiment, as shown in FIG. 20C, the second expandable member 109 may have a rigid section 103 or stiffening element to completely prevent any inflation in a direction opposite that of the at least one tubular insertion member 102. The rigid section 103 helps ensure inflation and deflection are more controlled and precise. Deflected tubular insertion member 102a and deflected radiation source position 128a are shown in dashed lines in FIG. 20C. It should be noted that FIGS. 20A-20C are exemplary only for simplicity of illustration herein and a plurality of tubular inflation members 102 may be used in conjunction with a second expandable member 109. In further embodiments, a plurality of tubular insertion members 102 may surround a second expandable member 109, such that inflation of expandable member 109 pushes or deflections each tubular insertion member 102 away from longitudinal axis 101.

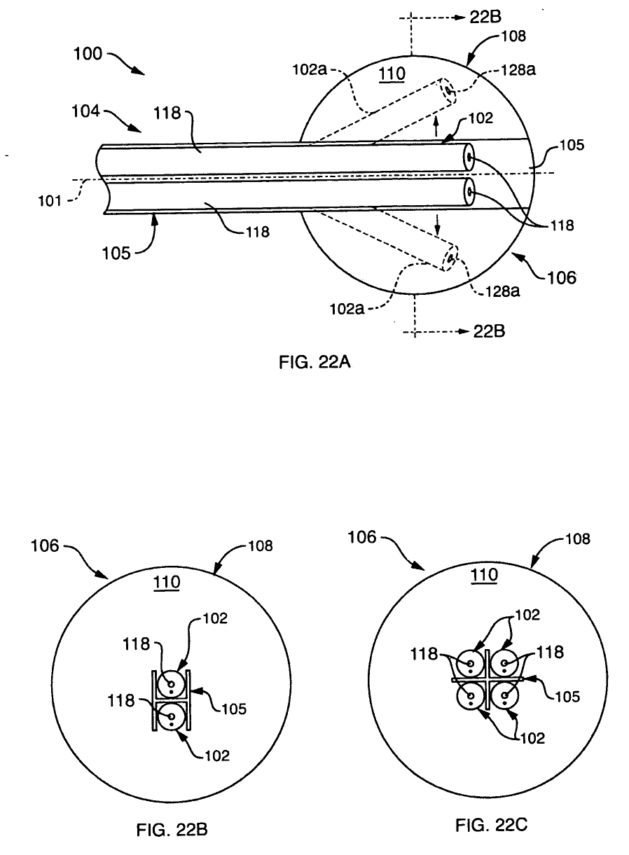

FIGS. 21A and 22A illustrate another brachytherapy treatment device 100 having a means for deflecting at least one tubular member 102 to deflect a radiation source position 128 to create an asymmetric radiation dosing profile to protect sensitive tissues 132. In this embodiment, an additional support structure 105 may be utilized so that the tubular member 102 is independently positionable within expandable member 108. The articulation or deflection of the tubular member 102 may be done using a number of different mechanisms, which will be described below in more detail.

As shown in FIGS. 21A and 22A, the brachytherapy treatment device 100 includes an insertion support structure 105, at least one tubular member 102, and an expandable member 108. The insertion support structure 105 has proximal 104 and distal 106 ends and provides support along the longitudinal axis 101 during insertion of the device 100 into a patient. The at least one tubular member 102 also has proximal 104 and distal 106 ends and is sized to be received by or fit within a portion of the insertion support structure 105. As shown in FIG. 21B, the insertion support structure 105 may partially surround and/or support tubular member 102. The at least one tubular member 102 has a radiation source lumen 118 extending along a longitudinal axis 101. The expandable member 108 defines an internal volume 110 and is disposed on and surrounds the distal end of the support structure 105 and the at least one tubular member 102.

With continuing reference to FIGS. 21A and 21B, the at least one tubular member 102 is adapted to be independently positionable with regard to the insertion support structure 105. The at least one tubular member 102 is capable of being articulated, bent, or deflected away from the longitudinal axis 101 of the support structure 105 to be positioned within the internal volume 110 to allow a wide range of flexible positioning options within expandable member 108. The articulation or deflection of the tubular member 102 within the expandable member 108 exposes the radiation source position lumen 118 to the internal volume 110 of the expandable member so that it may be positioned at any number of locations within the internal volume 110.

The at least one tubular member 102 may fit within insertion support structure 105 in a number of different configurations, which may depend upon the number of tubular members 102 utilized. For example, when one tubular member 102 is used, the insertion support structure may have an approximately C-shape or U-shape, as shown in FIGS. 21A and 21B. When two tubular members 102 are used, the insertion support structure 105 may have a shape similar to that of an I-beam and the tubular members 102 may rest between the ‘flanges’ of the I-beam, as shown in FIGS. 22A and 22B. When four tubular members 102 are used, the insertion support structure 105 may have a shape similar to that of an equilateral cross and the tubular members 102 may rest within each of the corners, as shown in FIG. 22C.

The radiation source position lumen 118 is configured to receive a radiation source, which may be disposed on the end of an articulating wire (coupled to an afterloader). The radiation source wire may be extended into the internal volume 110 at any depth and maneuvered into any position, thus the radiation source position 128 may be positioned at a wide range of differing positions within expandable member 108, providing a wide range of treatment planning options for patients.

Deflection or articulation of the tubular members 102 may be accomplished using a variety of different mechanisms, such as pull-wires 107 and/or shape memory materials. When using a shape memory alloy, such as nitinol, the tubular member 102 may be deflected to be offset from the longitudinal axis to provide an offset radiation source position, as shown in FIG. 22A.

Alternatively, as shown in FIG. 21A, the at least one tubular member 102 may be deflected via a pull-wire 107. The pull-wire 107 operably couples the proximal 104 and distal 106 ends of the tubular member 102 to provide control of the distal end 106 via the proximal end 104. The pull-wire 107 may be coupled to distal end 106 of tubular member 102 and operable to pull or deflect tubular member 102 away from longitudinal axis 101 of insertion support structure 105. Pull-wire 107 may be disposed within or along the length of tubular member 102 or insertion support structure 105. The pull-wire 107 may be coupled to a mechanism, such as a thumb-wheel, to provide adjustable control of the deflection of tubular member 102. With reference to FIG. 21A, deflected tubular member 102a is shown in dashed lines with an exemplary radiation source 128a disposed within and extending from radiation source lumen 118. In this embodiment, a radiation source may be extended any distance out of the radiation source lumen 118, providing a plurality of different radiation source position 128a options for creation of a variety of different asymmetrical radiation dosing profiles.

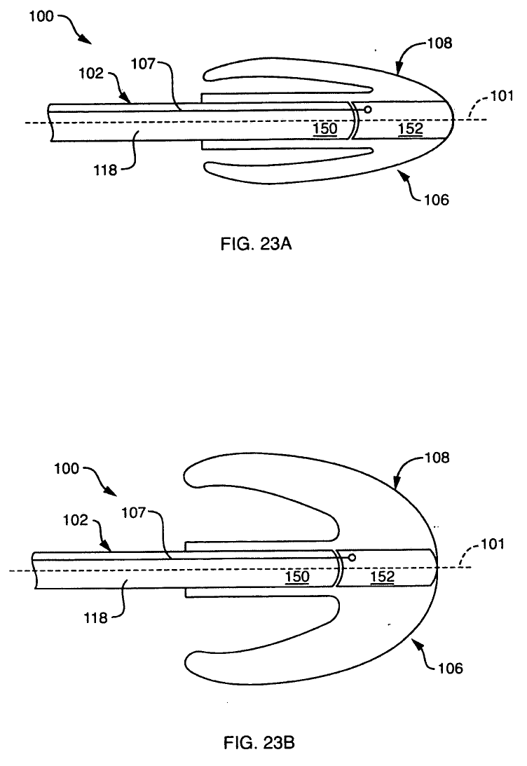

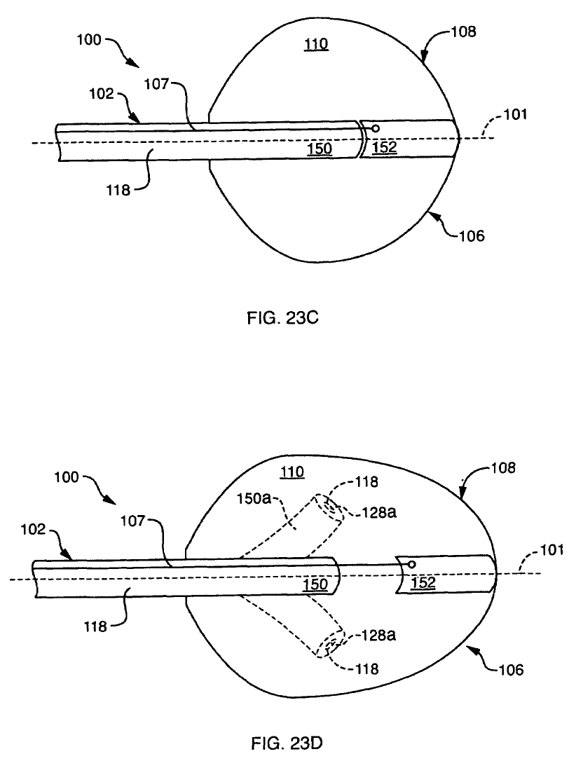

FIGS. 23A-23D illustrate yet another embodiment of a brachytherapy treatment device 100 having a means for deflecting the at least one tubular insertion member 102 to deflect a radiation source position 128 to create an asymmetric radiation dosing profile. In this embodiment, the at least one tubular insertion member 102 may have detachably mated proximal 150 and distal 152 tip segments, as will be described in detail below.

As shown in FIGS. 23A-23D, the brachytherapy treatment device 100 includes at least one tubular insertion member 102 and an expandable member 108. The at least one tubular insertion member 102 has a proximal end 104, a distal end 106, and a radiation source lumen 118 disposed along a longitudinal axis 101. The expandable member 108 is disposed on and surrounds the distal end 106 of the at least one tubular insertion member 102. The distal end 106 of the at least one tubular insertion member 102 has proximal 150 and distal 152 tip segments. The proximal 150 and distal 152 tip segments are in detachable mated engagement. A tensioning wire 107 may be utilized to couple the proximal 150 and distal 152 tip segments and to control detachment of the segments 150, 152. When the proximal 150 and distal 152 tip segments are detached, the radiation source lumen 118 is exposed to the interior volume of expandable member 108.

The ability to detachably mate proximal 150 and distal 152 tip segments allows the proximal 150 tip to articulate freely (shown in FIG. 23D) within the expandable member 108 when the expandable member 108 is inflated, while still providing a more rigid profile for easy insertion of the device 100 at a treatment site. During insertion, the tensioning wire 107 couples the proximal 150 and distal 152 tip segments to ensure they remain mated together during insertion to provide a more rigid insertion profile. During insertion, the expandable member 108 may also be in a folded or pleated state to present a more compact insertion profile, as shown in FIG. 23A. In this state, the expandable member 108 may be folded back onto itself along the longitudinal axis 101 of tubular insertion member 102 to minimize its insertion profile. However, once the device 100 is properly positioned at a treatment site, the expandable member 108 may be deployed (shown in FIGS. 23A-23D) and the proximal 150 and distal 152 tip segments may be detached by relaxing the tension on the tensioning wire 107 to separate or form an opening between the proximal 150 and distal 152 tip segments. Once the proximal 150 and distal 152 tip segments are separated, the tubular insertion member 102 is expanded or lengthened, as best shown in FIG. 23D. Once the brachytherapy treatment is completed, the tensioning wire 107 may again be tensioned to couple the segments 150, 152 back together for a more compact removal profile.

The detachment of proximal 150 and distal 152 tip segments may be accomplished by relaxing or lessening tensioning of the tensioning wire 107 or by breaking or severing the tensioning wire. In some embodiments, tensioning wire 107 may be designed to separate or detach once deployed. Tensioning wire 107 operably couples proximal 104 and distal 106 ends of the tubular member 102 so that distal end 106 may be operated via proximal end 104. Tensioning of wire 107 may be achieved by attaching wire 107 to a hub or port (not shown) on proximal end 104 of tubular insertion member 102. Tensioning wire 107 may be controlled by a number of different mechanisms. In one embodiment, tensioning wire 107 may be controlled by a thumb wheel or slide for adjustable tensioning. In another embodiment, tensioning wire 107 may be coupled to a tab which may be removed or released once the device 100 is in place to relax the tensioning wire 107 and detach segments 150, 152. In alternative embodiments, tensioning wire 107 may comprise any means for applying strain, pressure, tightness, tautness, stiffness, or rigidity to the proximal 150 and distal 152 tip segments to provide detachable coupling.

The detachment of proximal 150 and distal 152 tip segments creates a void or opening between the segments 150, 152, exposing the radiation source lumen 118 to the interior volume 10 of expandable member 108. The detachment of segments 150, 152 allows the proximal tip segment 150 to articulate freely while the expandable member 108 is inflated, as shown in FIG. 23D. Once the radiation source lumen 118 is exposed to the interior volume 110 of expandable member 108, a radiation source may be inserted through the radiation source lumen 118 and may be placed at any number of different radiation source positions (shown as 128a) within expandable member 108 via freely articulating proximal tip segment 150 (shown in dashed lines as 150a in deflected or articulating positions). The ability to steer or articulate proximal tip segment 150 provides a plurality of different radiation source positioning 128a options at a treatment site. In some embodiments, proximal tip segment 150 may have a number of different joints or segments to provide an improved range of articulating motion. Of course, some of these radiation source positions 128a may be offset from longitudinal axis 101 to create an asymmetric radiation dosing profile.

Methods for delivering brachytherapy treatment to a treatment site 112 in a patient are also provided herein. One exemplary method of performing brachytherapy treatment may commence with the placement of a brachytherapy treatment device or catheter 100 within a patient at a treatment site 112. The catheter 100 may comprise at least one tubular insertion member 102, as previously described above. Prior to placement of the catheter 100, it is common for a surgery to have been performed to remove as much of a tumor as possible. A surgical resection of the tumor is typically performed, leaving a surgical pathway and a resected space or cavity 130 for placement of the catheter 100 within the patient. In some embodiments, the placement of the catheter 100 may be done through an incision formed during removal of the tumor. In other embodiments, the placement of the catheter 100 may be done through a newly formed incision.

Once the catheter 100 is appropriately positioned within a patient, one or more expandable members 108 may be inflated, for example, to fill the cavity 130 of a resected tumor. The tissue 112 surrounding the cavity 130 may substantially conform to the outer surface of the outermost expandable member 108. In this manner, the tissue 112 surrounding the cavity 130 may also be positioned to reshape tissue to ensure a uniform boundary for the radiation dose profile and this may be utilized in conjunction with a deflected tubular insertion member 102 to achieve a predetermined asymmetrical radiation dosing profile.

A method of performing brachytherapy treatment may continue by deflecting the tubular insertion member 102. Deflecting, articulating, bending, shaping, or otherwise distorting the tubular insertion member 102 may be accomplished by altering wall thickness or material durometer or column strength of the tubular insertion member 102, activating or stimulating shape memory materials or a pre-stressed or pre-bent area, operating a pull-wire (such as via a thumb wheel or slide), operating a threaded member, pushing or pulling slidably sheath sections, inflating a second or third expandable member, detaching proximal and distal tip segments (such as via a pull-tab or wire), utilizing an insertion support structure to allow independent movement of the tubular insertion member 102, just to name a few examples.

The method then includes placing a radiation source at the radiation source position 128 at the treatment site 112. When the radiation source is placed the radiation dose profile 136 is reshaped (to 134) by the deflected shape of the tubular insertion member 102a. Following radiation treatment, the catheter 100 may remain within the patient's body in the treatment position so that it can be used during the next treatment session, or it may be removed.

Disclosed herein are devices and methods for use in treating proliferative tissue disorders by the application of radiation, energy, or other therapeutic rays. While the devices and methods disclosed herein are particularly useful in treating various cancers and luminal strictures, a person skilled in the art will appreciate that the methods and devices disclosed herein can have a variety of configurations, and they can be adapted for use in a variety of medical procedures requiring treatment using sources of radioactive or other therapeutic energy. These sources can be radiation sources such as radio-isotopes, or man-made radiation sources such as x-ray generators. The source of therapeutic energy can also include sources of thermal, radio frequency, ultrasonic, electromagnetic, and other types of energy.

It should be understood that various changes and modifications to the above-described embodiments will be apparent to those skilled in the art. The examples given herein are not meant to be limiting, but rather are exemplary of the modifications that can be made without departing from the spirit and scope of the described embodiments and without diminishing its attendant advantages.

What is claimed is:

1. A brachytherapy treatment device, comprising:
at least one tubular insertion member having a longitudinal axis, a proximal end and a distal end;
a first expandable member disposed on and surrounding the distal end of the tubular insertion member; and
means for deflecting the at least one tubular insertion member, wherein the distal end of the at least one deflected tubular insertion member within the first expandable member is offset from the longitudinal axis when deflected, the at least one deflected tubular insertion member configured to receive a radiation source to position a radiation source offset with regard to the longitudinal axis to form an asymmetric radiation dosing profile.

2. The device of claim 1, wherein the means for deflecting the at least one tubular insertion member comprises a differential wall thickness of a portion of a cross-section of the at least one tubular insertion member, wherein a portion of the cross-section of the at least one tubular insertion member has a width substantially thicker than a remaining portion of the cross-section.

3. The device of claim 2, wherein the portion of the cross-section of the at least one tubular insertion member having a substantially thicker width controls direction of deflection of the at least one tubular insertion member.

4. The device of claim 1, wherein the means for deflecting the at least one tubular insertion member comprises a threaded member operably coupling the distal and proximal end of the at least one tubular insertion member, wherein the threaded member is operable to compress the distal end of the at least one tubular insertion member to deflect the at least one tubular insertion member.

5. The device of claim 4, wherein the threaded member comprises a turnbuckle.

6. The device of claim 4, wherein the threaded member comprises a lead screw.

7. The device of claim 1, wherein the means for deflecting the at least one tubular insertion member comprises a pre-stressed tubular insertion member.

8. The device of claim 1, wherein the means for deflecting the at least one tubular insertion member comprises a portion of the at least one tubular insertion member formed of a material having a first durometer and a material having a second durometer, wherein the material having a first durometer and the material having a second durometer have different durometers.

9. The device of claim 8, wherein a meeting point of the materials having first and second durometers creates a fulcrum for deflecting the at least one tubular insertion member.

10. The device of claim 1, wherein the means for deflecting the at least one tubular insertion member comprises a shape memory material.

11. The device of claim 1, wherein the means for deflecting the at least one tubular insertion member comprises a second expandable member disposed within the first expandable member, the second expandable member positioned adjacent the first expandable member and the at least one tubular insertion member and operable to deflect at least one tubular insertion member when inflated.

12. The device of claim 1, wherein the means for deflecting the at least one tubular insertion member comprises a slide mechanism.

13. A brachytherapy treatment device, comprising:
at least one tubular insertion member having a longitudinal axis, a proximal end and a distal end; and
an expandable member disposed on and surrounding the distal end of the at least one tubular insertion member;
wherein the distal end of the at least one tubular insertion member within the expandable member has a substantially helical shape, the at least one helical-shaped tubular insertion member operable to receive a radiation source to position a radiation source offset with regard to the longitudinal axis to form an asymmetric radiation dosing profile.

14. A brachytherapy treatment device, comprising:
at least one tubular insertion member having a proximal end, a distal end, and a radiation source lumen disposed along a longitudinal axis; and
an expandable member disposed on and surrounding the distal end of the at least one tubular insertion member;
wherein the distal end of the at least one tubular insertion member within the expandable member has proximal and distal tip segments, the proximal and distal tip segments in detachable mated engagement, wherein detaching the proximal and distal tip segments exposes the radiation source lumen of the at least one tubular insertion member to an interior volume of the expandable member, wherein the radiation source lumen is adapted to receive and position a radiation source within the interior volume of the expandable member to form an asymmetric radiation dosing profile.

15. The device of claim 14, further comprising a wire disposed within the at least one tubular insertion member and operably coupling and detachably mating the proximal and distal tip segments, wherein operation of the wire at the proximal end controls deflection of the at least one tubular insertion member at the distal end.

16. A brachytherapy treatment device, comprising:
an insertion support structure having a proximal end and a distal end;
at least one tubular member having proximal and distal ends and sized to be received by the insertion support structure, the at least one tubular member having a radiation source lumen extending along a longitudinal axis; and
an expandable member defining an internal volume, the expandable member disposed on and surrounding the distal end of the at least one tubular member;
wherein the at least one tubular member is adapted to be independently positionable with regard to the insertion support structure, the at least one tubular insertion member deflectable within the internal volume to expose the radiation source lumen to the internal volume to form an asymmetric radiation dosing profile.

17. The device of claim 16, wherein the at least one tubular member is positionable via a pull-wire disposed within the at least one tubular member, wherein operation of the pull-wire at the proximal end controls deflection of the at least one tubular member at the distal end.

18. The device of claim 16, wherein the at least one tubular member is positionable via a shape memory material.

19. A radiation treatment device comprising:
a tubular member having a longitudinal axis, a distal portion adapted to be inserted within a patient to a treatment site and a proximal portion adapted to extend out of the patient;
an expandable device disposed on said distal portion of said tubular member, said expandable device configured to be expanded such that tissue conforms to an outer surface of the expandable device whereby such conforming tissue defines an inner boundary of target tissue to be treated by radiation; and
a radiation source position located within said tubular member at a position axially corresponding to said first and second expandable devices; and
an adjustable radiation source position mechanism for controllably adjusting the position of the radiation source position.

20. The device of claim 19, wherein said adjustable radiation source position mechanism comprises a plurality of pull wires having a distal end operably coupled to said distal portion of said tubular member and proximal end extending proximally to said proximal portion of said tubular member.
